(19) United States
(12) Patent Application Publication
(10) Pub. No.: US 2013/0233312 A1
(43) Pub. Date: Sep. 12, 2013

(54) Portable Sublimation Device

(71) Applicant: Harris Cohn, Columbus, OH (US)

(72) Inventor: Harris Cohn, Columbus, OH (US)

(21) Appl. No.: 13/766,370

(22) Filed: Feb. 13, 2013

Related U.S. Application Data
(60) Provisional application No. 61/609,121, filed on Mar. 9, 2012.

(51) Int. Cl. A61M 15/06

(52) U.S. Cl. CPC A61M 15/06
USPC 128/202.21

(57) ABSTRACT

A device for sublimating a chemical that has a boiling point that is lower than the combustion temperature of a substance containing the chemical comprising a vaporization tube, a mouthpiece stem, a holder and a heating device.

### CROSS-REFERENCES TO RELATED APPLICATIONS

This patent application claims the benefit of provisional patent application Ser. No. 61/609,121, filed Mar. 9, 2012, the complete subject matter of which is hereby incorporated herein by reference in its entirety.

### FIELD OF THE INVENTION

The present invention relates generally to devices that release chemicals from a substance without creating smoke and more particularly to a portable apparatus for the sublimation of certain chemicals that have boiling points lower than the combustion temperature of the substance.

### BACKGROUND OF THE INVENTION

Sublimation is the process of a changing a solid compound directly to a gaseous vapor without converting the solid to a liquid in the process. In that the gaseous vapor is produced at lower temperatures than it takes to burn the material containing the compound, no smoke or toxic chemicals are produced.

When the solid compounds are medications or drugs, the vaporized compounds can be inhaled to introduce the compound into a person's body. Specifically, as an alternative to smoking, sublimation of nicotine from tobacco and THC from marijuana reduces the amount of carbon monoxide, tar and gaseous combustion toxins generated by smoking.

According the a 2001 study in Respiratory Care in Clinics of North America, inhalers are second only to pills as the most common medication form in the world. Making vaporized drugs available to patients would provide advantages. For example, lung cancer is difficult to treat. Cisplatin is a chemotherapy drug currently prescribed to treat lung cancer, but can cause serious complications, such as kidney damage, when administered intravenously. Inhalation of vaporized cisplatin would allow direct treatment of diseased tissue while avoiding harmful side effects.

While some portable vaporizers exist, most vaporizers are typically a table top device requiring an electrical source. Portable vaporizers currently on the market are either electric (corded or battery) or butane powered. Electric portable vaporizers require some sort of NIMH battery whether enclosed, detachable, or a simple rechargeable AA or AAA NIMH battery. The problem with these portable vaporizers is that the batteries take an average of 16 hours to re-charge and the batteries discharge rapidly due to the nature of this application. These vaporizers also typically take roughly 5 minutes to heat up before each individual inhalation. Where a vaporized drug is used to treat sudden acute symptoms, immediate delivery is essential. Butane powered vaporizers require the user to fill a butane reservoir with butane gas using cans of butane which are difficult to find, expensive, and toxic. Because these vaporizers are portable, the butane reservoirs are generally so small that they need to be refilled after only several uses. Additionally, when using butane-powered vaporizers, the user inhales butane gas, which is toxic, and in many cases causes nausea, headaches, and in some sever cases can cause narcosis, asphyxia, and cardiac arrhythmia among other life threatening conditions. Current vaporizers are also expensive—ranging up to $500 for a non-medical device.

Medical inhalers may be hand held and portable, but many different devices exist—causing difficulties for users by their complexity. Pressurized metered-dose inhalers requires a slow inspiratory flow rate, whereas a dry powder inhaler requires rapid inhalation. The type of inhaler can substantially reduce the delivery and effectiveness of the medication.

It is an object of the present invention to provide an inexpensive and simple novel device and method for producing vaporized chemicals for inhalation.

Another object of this invention is to provide a portable device that is able to vaporize a compound within seconds rather than minutes.

### SUMMARY OF THE INVENTION

The present invention is directed to a portable device for sublimating a chemical that has a boiling point that is lower than the combustion temperature of a substance containing the chemical. The device comprises a vaporization tube, a mouthpiece stem and a holder.

The tube is generally straight pipe or tube with a uniform diameter that is formed from a borosilicate glass or similar material. The tube has a closed end and an open end. The closed end may be tapered or rounded or squared. the closed end may be formed as part of the tube, or may comprise a removable cap to aid in clean out of spent compounds.

The mouthpiece stem is a generally straight elongated cylinder having a diameter that is less than the diameter of the tube and a length that is less than a length of the tube. The stem is formed from a borosilicate glass or similar material. The stem has a first end that is opened and is flanged. The flange is slightly larger than the open end of the tube and provides a mechanism for a user to grasps the stem and pull it out of the tube when inserted. The other end of the stem comprises an open elongated narrowed section. The narrowed section has a diameter that is about ⅓ the diameter of the stem. When not in use, the stem fits into the tube with the flange against the open end of the tube.

The holder is comprised of a polyolefin material sealed onto a segment of an outside surface of the vaporization tube near the open end. A portion of the holder extends slightly beyond the open end of the tube to create a fitting. The polyolefin material of the fitting is slightly flexible and has a diameter smaller than the tube opening and larger than the cylinder segment of the stem. The fitting is uniform in diameter except for an indent formed at point along the circumference. The stem fits snugly against the fitting, with the indent allowing air to flow from the outside of the device into the tube, through the narrowed end of the stem when a user creates a suction on the flanged end.

In use, a user places a substance containing the chemical to be sublimated in the closed end of the tube. this can be done through the open end and the substance tapped down, or via the closed end if the cap version is used. The user then inserts the narrowed end of the stem into the open end of the tube and heats the substance to sublimate the chemical. When vapor begins to rise from the compound (typically within a few seconds), the user places his lips over the flange and inhales the sublimated chemical.

As used herein, “approximately” means within plus or minus 25% of the term it qualifies. The terms approximately and “about” means between 112 and 2 times the term it qualifies.

The devices and methods of the present invention can comprise, consist of, or consist essentially of the essential elements and limitations of the invention described herein, as well as any additional or optional ingredients, components, or limitations described herein or otherwise useful in compositions and methods of the general type as described herein.

Numerical ranges as used herein are intended to include every number and subset of numbers contained within that range, whether specifically disclosed or not. Further, these numerical ranges should be construed as providing support for a claim directed to any number or subset of numbers in that range or to be limited to the exact conversion to a different measuring system, such, but not limited to, as between inches and millimeters.

All references to singular characteristics or limitations of the present invention shall include the corresponding plural characteristic or limitation, and vice versa, unless otherwise specified or clearly implied to the contrary by the context in which the reference is made.

All combinations of method or process steps as used herein can be performed in any order, unless otherwise specified or clearly implied to the contrary by the context in which the referenced combination is made.

Terms such as “top,” “bottom,” “right,” “left,” “above”, “under”, “side” “front” and “back” and the like, are words of convenience and are not to be construed as limiting.

### BRIEF DESCRIPTION OF THE DRAWINGS

FIG. 1 is a perspective view of an embodiment of the assembled device.

FIG. 2 is a perspective view of the stem piece removed from the tube.

FIG. 3 is an end view of the tube showing the holder.

FIG. 4 and FIG. 5 are perspective views of the holder.

FIG. 6 is a perspective view of the stem piece removed from the tube.

FIG. 7 is a perspective view of the vaporization tube.

FIG. 8 is a perspective view of the stem piece in the tube with the holder attached.

FIG. 9 and FIG. 10 are perspective views of the holder attached to the tube.

FIG. 11 is a perspective view of the stem piece in the tube with the holder attached.

### DETAILED DESCRIPTION OF THE INVENTION

Reference will now be made in detail to the exemplary embodiments of the invention, examples of which are illustrated in the accompanying drawings. In accordance with an embodiment of the present invention, a simple portable device that quickly sublimates a chemical from a substance is provided.

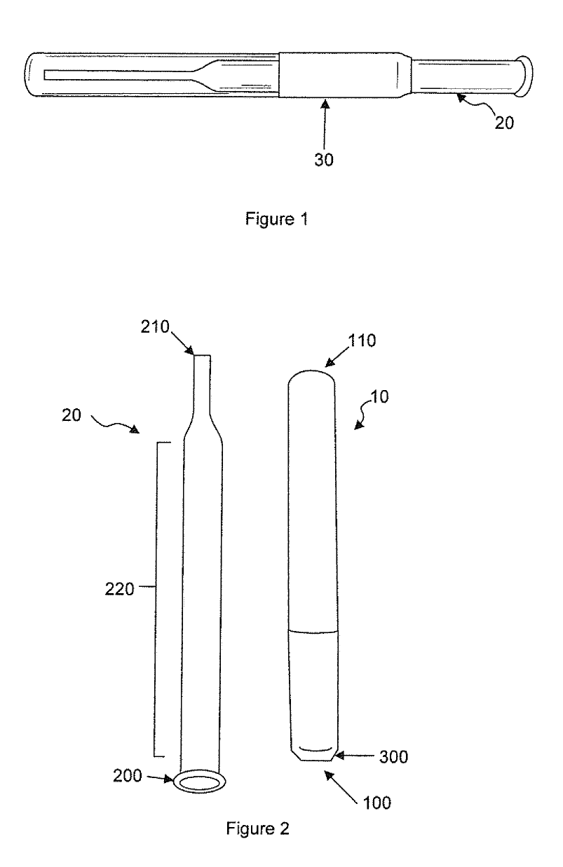

As shown in an embodiment depicted in FIG. 1, the vaporizer comprises at least three parts: a vaporization tube 10, a mouthpiece stem 20, and a holder 30. The vaporization tube and mouthpiece stem are made from any chemical and thermal resistance substance, such as ceramic, glass and glass-like materials that have a low coefficient of thermal expansion. In an embodiment, the vaporization tube and mouthpiece stem are formed from borosilicate glass. The holder is formed from any high temperature and flame resistance insulating material. In an embodiment, the holder is a mechanically expanded extruded plastic tube made of nylon or polyolefin that shrinks when heated to return the tube to the original diameter as when it was extruded.

As shown in FIG. 2, the tube 10 is tube generally straight with a uniform diameter and comprises an open end 100 and a closed end 110. The mouthpiece stem 20 is a generally straight elongated cylinder open on both ends. The central part of the cylinder has a diameter that is less than the diameter of the tube. The length of the mouthpiece is less than the length of the tube. A first end 200 of the stem is opened into a flange and the second end 210 has a diameter that narrows to a diameter that is from about ½ to about ⅓ the diameter of the central part 220 of the stem. The flange has an outside diameter larger than the diameter of the tube. In an embodiment, the narrowed open end 210 may comprise additional openings, such as slots in the sides of the narrowed opening (not shown).

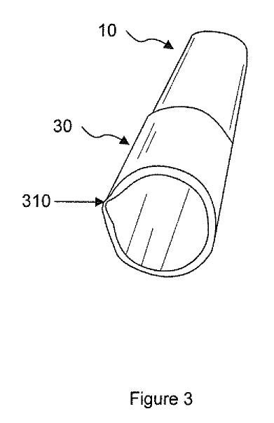

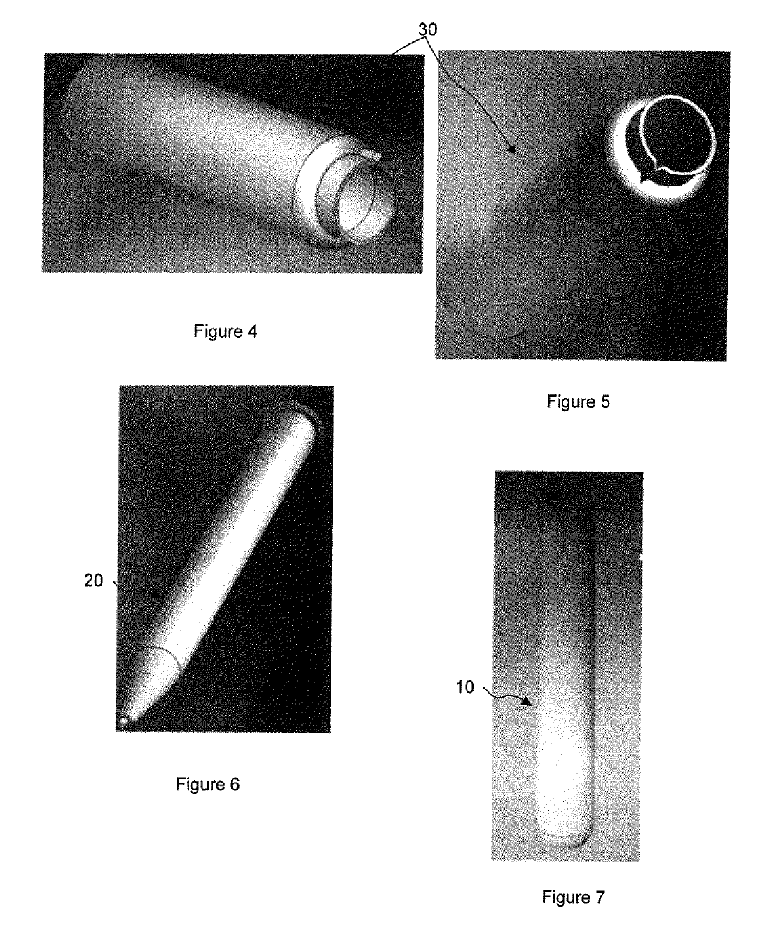

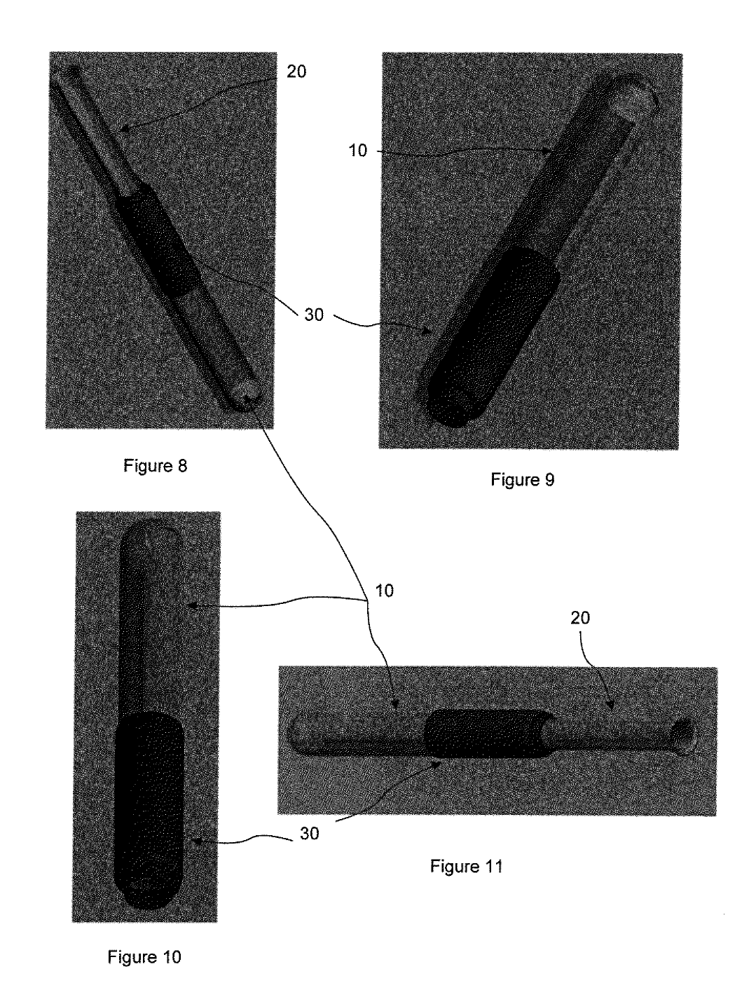

The holder 30 is an open sleeve that is sealed onto a segment of an outside surface of the vaporization tube at the open end. A portion of the holder extends beyond the end of the tube to create a fitting 300. The fitting has an end diameter smaller than the diameter of the tube, but larger than the diameter of the central portion of the stem such that the stem fits snugly against the fitting. The fitting is formed with an indent 310. The indent 310 (best seen in FIG. 3) prevents a complete seal between the tube and the stem and allows for the flow of air from outside the tube to be drawn into the tube and into the narrowed end of the stem when suction is applied to the flange end of the stem.

In an embodiment, existing parts are used to assemble the device of the present invention. In this embodiment, the device is assembled quickly without tooling or extruding the parts. Here, a 4 mm test tube (such as Fisherbrand Disposable Borosilicate Glass Tubes with Plain End—catalog no. 14-961-25 available from Fisher Scientific) is used for the tube, and a 3 inch 2 ml pipette (such as Fisherbrand Glass Medicine Dropper—catalog no, 14-955-215 available from Fisher Scientific) is used for the stem. A piece of heat shrinkable tubing with a shrink ratio of about 2:1 (such as VERSAFIT-3/8-0-SP available from RayChem Tubing) is placed over the tube with the stem inserted and heated to shrink the plastic to conform to the tube and form the fitting. A small metal rod is placed at a point between the stem and the tube to form the indent.

As assembled, the narrowed end of the stem is inserted into the tube with the flange resting against the open end of the tube. The assembled device can be easily carried in a pocket or purse. To use the device, a user removes the stem and places a substance containing a chemical to be sublimated in the closed end of the tube. The substance is placed in the tube near the closed end via the open end. Less than about 1 g of substance is used, preferably less than about 0.1 g of substance is used. In an embodiment, about 0.06 g of substance is used. An advantage of the present invention is the small amount of substance that is used. The user then inserts the narrowed end of the stem into the open end of the tube, holds the tube with a finger and a thumb of one hand at the holder and applies a heat source (not shown) to the portion of the tube containing the substance. The heat source is any portable heating device, such as a chemical warmer, electrical coil, heater and the like, that provides instant heat at a temperature of about 300° F. to about 600° F. In an embodiment, the heat source is an open flame from a disposable lighter. The heat source causes the chemical to change from a solid to a gas within about 5 seconds without generating enough heat to combust the substance. The heat source is removed, and the user places his/her lips over the flange and inhales the sublimated chemical. When the vapor production ceases, the used substance is removed from the tube and the device is ready for the next sublimation.

One skilled in the art would understand that any structure could be used without departing from the spirit and scope of the invention. Thus, the width and length need not be equal and can be varied, so long as device provides sufficient length to prevent the user from burning when the flame is applied. While a circular tube and stem is preferred, any shape that allows the tube and the stem to function as described can be employed.

The foregoing descriptions of specific embodiments and examples of the present invention have been presented for purposes of illustration and description. They are not intended to be exhaustive or to limit the invention to the precise forms disclosed, and obviously many modifications and variations are possible in light of the above teachings. It will be understood that the invention is intended to cover alternatives, modifications and equivalents. The embodiments were chosen and described in order to best explain the principles of the invention and its practical application, to thereby enable others skilled in the art to best utilize the invention and various embodiments with various modifications as are suited to the particular use contemplated. It is therefore to be understood that within the scope of the appended claims, the invention may be practiced otherwise than as specifically described herein.

I claim:

1. A portable device for sublimating a chemical that has a boiling point that is lower than the combustion temperature of a substance containing the chemical comprising:
a vaporization tube, the tube generally straight with a uniform diameter, formed from a borosilicate glass, and having a closed end and an open end;
a mouthpiece stem, the stem generally straight elongated cylinder having a diameter that is less than the diameter of the tube and a length that is less than a length of the tube, the stem formed from a borosilicate glass and having a first end that is opened into a flange and a second end that narrows to a diameter that is about ⅓ the diameter of the stem, such that, when not in use, the stem telescopes into the tube with the flange against the open end of the tube; and
a holder, the holder comprised of a polyolefin material sealed onto a segment of an outside surface of the vaporization tube at the open end, a portion of the holder extending beyond the open end of the tube to create a fitting; the fitting having a diameter smaller than the tube and larger than the cylinder segment of the stem and formed with an indent such that the stem fits snugly against the fitting, but the indent prevents a seal between the tube and the stem.

2. A method of using the device of claim 1 wherein a user:
a) places the substance containing the chemical in the closed end of the tube via the open end.
b) inserts the narrowed end of the stem into the open end of the tube,
c) heats the substance to sublimate the chemical;
d) places his lips over the flange and inhales the sublimated chemical.
